(19) United States
(12) Patent Application Publication
(10) Pub. No.: US 2004/0064167 A1
(43) Pub. Date: Apr. 1, 2004

(54) Dermatological Treatment Apparatus and Method

(76) Inventors: Andrew J. Berry, Cheshire (GB); Andrew Charlton, Cheshire (GB); Jonathan Exley, Cheshire (GB); Benjamin Dickinson, Cheshire (GB)

Correspondence Address: TAYLOR RUSSELL & RUSSELL, P.C., 4807 SPICEWOOD SPRINGS ROAD, BUILDING ONE, SUITE 1200, AUSTIN, TX, 78759, US

(21) Appl. No.: 10/604,085

(22) Filed: Jun. 25, 2003

(30) Foreign Application Priority Data
Jun. 28, 2002 (GB) 0214984.7

(51) Int. Cl. A61N 1/00

(52) U.S. Cl. 607/89

(57) ABSTRACT

Dermatological treatment apparatus is disclosed. The treatment apparatus comprises a treatment head for generating intense pulses of light to an area of skin under treatment. Most typically, the treatment head generates pulses by a high-intensity source of non-coherent light, such as a flashtube. The apparatus includes a controller for controlling operation of and for driving the treatment head. A sensing means is associated with the treatment head for detecting a condition of an area of skin under treatment. The sensing means is operative to send a signal to the controller indicative of the detected condition, and the controller being operative to modify operation of the treatment apparatus in response to that signal. Most typically, the control unit selects initial parameters for a treatment operation, and these parameters are subsequently modified in response to signals received from the sensing means.

### CROSS REFERENCE TO RELATED APPLICATIONS

This application claims foreign priority benefits under 35 U.S.C. 119 of United Kingdom application number 0214984.7, filed Jun. 28, 2002.

### BACKGROUND OF INVENTION

This invention relates to dermatological treatment apparatus. In particular, it relates to dermatological treatment apparatus that operates by application of high-intensity flashes of non-coherent light to an area of skin that is to be treated.

### SUMMARY OF THE PRIOR ART

Treatment of skin by application of coherent laser light to achieve clinical or cosmetic effects has been known for many years. The efficacy and limitations of such treatment are well understood. However, the cost of the apparatus and the level of skill required to conduct such treatment has limited the extent of its application.

In order to increase the availability of dermatological treatment, particularly (but not exclusively) in cosmetic applications, an alternative treatment method has been developed that uses the application of non-coherent light typically generated in pulses by a flash tube. This type of treatment may be referred to generally as “intense pulsed light” treatment. This method of treatment can be performed using apparatus that is considerably less costly than laser treatment apparatus, and has the further advantage of being able to treat a larger skin area in a given time than equivalent laser treatment methods. Thus, it is being practiced in an ever-wider range of circumstances by a broad range of practitioners.

### SUMMARY OF INVENTION

Contrary to received understanding, the present applicants have realised that the factors that contribute to successful treatment with intense pulsed light are very different to those that contribute to successful laser treatment, and that many of the principles that are successfully applied to laser treatment cannot be directly adapted. Wider application of intense pulsed light treatment has also brought about further problems that are not present in laser treatment. In particular, this type of treatment has the potential to introduce a large amount of energy into the skin, which can result in a potentially damaging temperature rise in the dermis. If this is not controlled, it could lead to damage such as blistering. While laser treatment can also give rise to heat build-up, it is normally performed by a highly skilled and well-trained person who will be aware of the risks and the steps necessary to avoid them. On the other hand, intense pulsed light treatment may be performed by people of lower skill, and may therefore be less well controlled.

An aim of this invention is to provide intense pulsed light dermatological treatment apparatus that is less likely to give rise to heat damage than conventional intense pulsed light treatment apparatus. However, it is also important not to subject the skin to excessive cold.

Therefore, from a first aspect, this invention provides dermatological treatment apparatus comprising:

a treatment head for generating intense pulses of light to an area of skin under treatment;

a controller for controlling operation of and for driving the treatment head in accordance with one or more treatment parameters;

a sensing means associated with the treatment head for detecting a condition of an area of skin under treatment; wherein

the sensing means is operative to send a signal to the controller indicative of the detected condition, and the controller being operative to modify one or more treatment parameters in response to that signal.

Thus, when the controller determines that there is a risk that damage might occur at an area under treatment through its analysis of the signal from the sensing means, it modifies the parameters to minimise or remove the risk that damage does, in fact, happen.

More specifically, the controller selects values of one or more control treatment parameters, and one or more of those parameters are modified during treatment in response to a signal received from the sensing means.

The principal cause of damage to skin during intense pulsed light treatment is build-up of excessive heat in the dermis. As a result of intense pulsed light treatment applying energy to a comparatively large area of skin, build-up of heat may be more of a problem in this type of treatment than it is in laser treatment. Therefore, in a typical embodiment of the invention, the sensing means is operative to detect heat in the area under treatment. Detection of heat may be carried out by measurement of the temperature of the surface of the skin. Most preferably, this is done by way of a passive measuring device that does not introduce energy into the skin. For example, the measuring device may detect and measure infrared radiation emitted by the skin for example by way of a thermocouple. More specifically, infrared light is collected by a lens that focuses it onto some material that absorbs it. The temperature of this material is then measured by a number of thermocouples. In this way, temperature measurement is non-contact with respect to the skin under treatment. Most advantageously, the temperature sensor has low sensitivity to the light of the treatment pulse. This ensures that the treatment and detection functions of the apparatus do not interact with one another.

It must be realised that such a measurement gives information about the temperature of the epidermis (which is a poor conductor of heat), and only indirect measurement of the temperature of the dermis below. Therefore, there may be better (or at least complimentary) measurements that can be made in order to deduce the temperature of the dermis. For example, the sensing means may be operative to measure optical properties of the skin.

In order to ensure that the energy applied during treatment is correctly controlled, it is desirable to determine properties of the skin. For example, the colour of the skin may be determined to establish the skin type (e.g. on the Fitzpatrick Scale), which is a measure of the amount of melanin it contains. This allows the system to appropriately set up the initial parameters needed for a treatment. The colour may be measured by making measurement of the intensity of light reflected by the skin at several discrete frequencies. For example, it may measure one or more of blue, green, red and infrared light. In order to measure colour, a source of light must be applied to the skin and an analysis made of the light that the skin reflects. In preferred embodiments of the invention, reflection of the intense light pulses that perform the treatment is analysed to determine colour. This is advantageous in that it does not complicate the apparatus by requiring an additional source of light to be provided, and in that the measurement does not add to the amount of energy being delivered to the skin.

Additional information about the condition of the skin may be obtained by monitoring the size of blood vessels in the skin undergoing both to set initial treatment parameters and to modify those parameters, if appropriate, during the course of treatment.

A most basic response to detection of unfavourable conditions developing in the skin is for the controller to modify treatment parameters to reduce (possibly entirely) the amount of energy that is being applied to the skin. For example, the modified parameters include one or both of intensity of applied light pulses and repetition period of application of light pulses. However, this is somewhat counterproductive in that it defeats the basic object of the apparatus. More preferably, the apparatus is provided with cooling means to increase the rate at which energy is removed from the skin. Therefore, as an alternative or an additional response to the onset of unfavourable conditions, the controller can operate the cooling means. In a simple embodiment, the cooling means may include a blower for passing a stream of cold air over the area being treated. It is common for intense pulsed light treatment apparatus to include a contact component, such as a light guide, that makes contact with the skin during treatment. The cooling means may be applied to the contact component. For example, fluid passages may be provided through and/or around it. Alternatively or additionally, a Peltier cooler may be applied to it. In such embodiments, the contact component (in particular, a light guide) may advantageously be formed of quartz, which has high thermal conductivity.

In intense pulsed light treatments, it is common to apply a transparent compound, such as a clear gel, to the area of skin prior to treatment. This serves to provide an efficient optical pathway between the treatment head and the skin. Apparatus embodying the invention may be provided in combination with an optical compound, which compound is at a temperature below that of ambient temperature and/or below the temperature of the skin prior to commencement of treatment. This has several effects that contribute to making use of such a compound surprisingly effective. Clearly, the heat flows from the skin to the compound because the compound is at a lower temperature than the skin. The compound may also increase thermal conduction through the epidermis. Heat can also travel through the compound to an area that greater than the area of the treatment head, allowing heat to radiate from the area of the compound.

The invention has particular application to treatment apparatus in which the treatment head includes a flashtube for generating pulses of light.

From a second aspect, the invention provides a dermatological treatment method in which a treatment head is used for generating intense pulses of light to an area of skin under treatment; a controller controls operation of and drives the treatment head; a sensing means associated with the treatment head detects a condition of an area of skin under treatment; and the sensing means sends a signal to the controller indicative of the detected condition, and the controller modifies operation of the treatment apparatus in response to that signal.

### BRIEF DESCRIPTION OF DRAWINGS

FIG. 1 is a general schematic view of apparatus being a first embodiment of the invention;

FIG. 2 is a flow diagram showing a basic sequence of operation of a system embodying the invention; and

FIG. 3 is a flow diagram of a treatment cycle in a treatment apparatus embodying the invention.

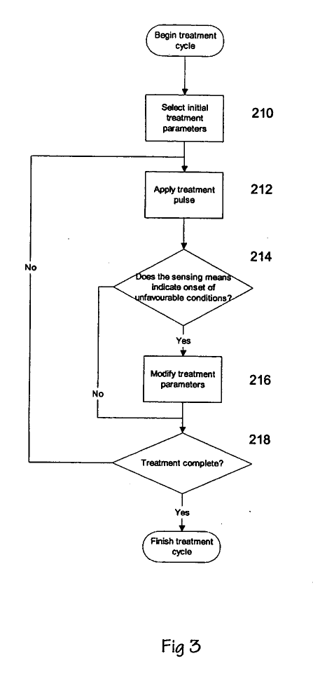

### DETAILED DESCRIPTION

An embodiment of the invention will now be described in detail, by way of example, and with reference to the accompanying drawings, in which:

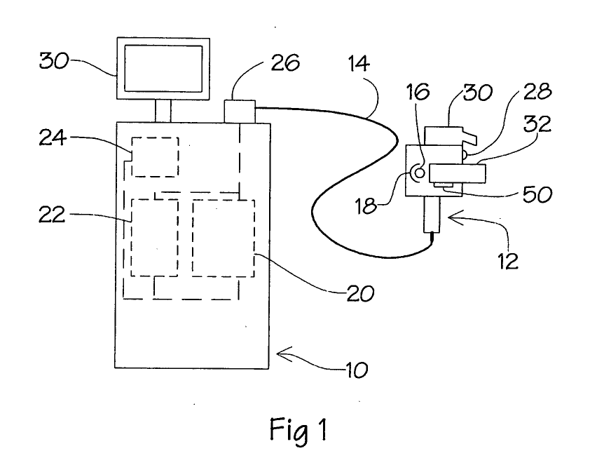

With reference first to FIG. 1, a dermatological treatment apparatus embodying the invention is shown in a condition ready for use. The apparatus comprises a control unit 10, a treatment head 12 and an interconnecting cable 14 that connects the treatment head 12 to the control unit 10.

The control unit 10 includes an electrical power supply stage 20, a coolant supply stage 22 and a control stage 24. The control stage 24 includes a computer system that executes a control program to control operation of the apparatus, and in particular, the power supply stage 20 and the coolant supply stage 22. Interaction between a user and the control stage 24 is handled by a touch screen display 30. The control stage 24 further includes a memory 102 that contains a control program, calibrations and other data and/or parameters relating to various treatment heads, and data relating to the treatment heads that are immediately available to a user.

The treatment head 12 is a high-intensity pulsed non-laser light treatment head. The treatment head includes a flashtube 16 that is supplied with pulses of electrical power and a supply of cooling water by, respectively, the power supply stage 20 and the coolant supply stage 22. The flashtube 16, in operation, delivers pulses of light that are reflected by a reflector 18 through a waveguide 32 to a treatment area of the skin.

An infrared detector 28 is provided on the treatment head, positioned to have a field of sensitivity that is within the treatment area. The infrared detector 28 is a non-contact infrared temperature sensor that generates an electrical output signal that is indicative of the temperature of the surface on which it is directed.

An air blower 30 is also carried on the treatment head 12. The air blower can be operated to generate a stream of air that is directed onto the area under treatment.

The cable 14 interconnects the control unit 10 and the treatment head 12. The cable 14 contains a plurality of electrical conductors to convey electrical power to components of the treatment head 12 and return signals from the infra-red detector 28, and a plurality of fluid conduits to carry cooling fluid (for example, water) circulating between the control unit 10 and the treatment head 12. The cable 14 is connected to the control unit by a plug 26 that engages with a socket provided externally on the control unit 10. The plug 26 and socket provides electrical and fluid interconnection between the control unit 10 and the cable 14. In addition, the plug 26 provides a data connection to the control unit, various arrangements of which will be discussed below. The treatment head 12, the interconnecting cable 14 and the plug 26 will be referred to as a treatment head assembly.

The control stage 24 interacts with a user by means of the touch screen 30. This touch screen 30 presents a graphical user interface by means of which a user can control operation of the apparatus by touching regions of the touch screen to perform functions indicated by a display rendered on the touch screen 30.

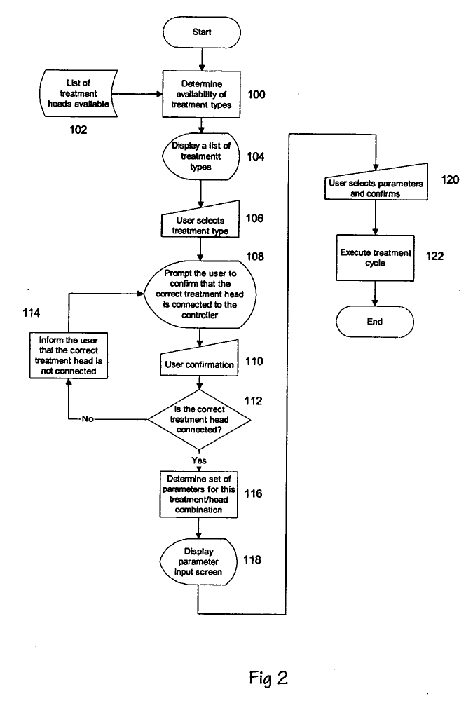

With reference now to FIG. 2, a sequence of operation of the apparatus described above will now be described.

When a user first starts a treatment operation, the control unit first generates a list of treatment types available (Step 100). This is achieved by referring to the memory 102, and determining the range of treatments that can be carried out with the selection of treatment heads that are available. This list of treatment types is then displayed as a list (Step 104) on the display 30. A user can then select a treatment type (Step 106) by touching an appropriate region of the display 30.

The user is then prompted (Step 108) to confirm that the correct treatment head 12 is connected to the control unit 10. Once the user makes such confirmation (Step 110) the control stage 24 determines (Step 112) the identity of any treatment head 12 that is connected to the control unit 10. If no treatment head 12 is connected, or an incorrect treatment head is connected, the control stage 24 causes an appropriate warning message to be displayed (Step 114) and then once again prompts the user to confirm that the correct treatment head is connected (Step 108).

The control stage 24 then retrieves data from its memory 102 that specifies the operating parameters and the range of values of each of the parameters appropriate to the connected treatment head 12 (Step 116). These parameters may include, amongst others, skin type, hair colour, beam fluence, beam spot size, pulse length, the number of pulses in a pulse train to be applied, and the temporal spacing between pulses in the pulse train. The control stage 24 also selects a default set of parameters and an allowable range for each of these parameters. This information is then used by the control stage 24 to generate an input screen on the display 30. The default set of parameters is initially displayed in the input screen, and the user can use the input screen to vary these parameters within the allowable range as required for the treatment cycle at Step 120.

Once the user has confirmed their selection, the treatment cycle can be commenced (Step 122). The user first applies a cooled transparent gel to the area to be treated. The waveguide of the treatment head is then brought into contact with the gel, and the user operates a control to initiate the delivery of treatment pulses. The principle purpose of the gel is to provide a predictable light path from the waveguide to the skin under treatment. Additionally, the gel is cooled prior to application to cool the skin prior to treatment. Additionally, the gel can provide a thermally conductive path to assist in the escape of heat from the area under treatment.

The electrical energy delivered to the treatment head 12 is initially determined by the control stage by selecting initial parameters (Step 210) that are stored in its memory 102 and are appropriate to the physical properties of the connected treatment head 12. The control unit 10 then causes the treatment head to generate pulses as defined by the parameters (step 210). However, during the course of the treatment cycle, if signals from the sensing means indicate that unfavourable conditions are arising (step 214) the parameters are modified by the control unit (step 216) in response to the condition of the area under treatment. This is repeated until the treatment cycle is complete (step 218).

Specifically, the control stage 24 periodically receives and analyses a signal from the infrared detector 28, and from this analysis, determines the temperature of the area under treatment. While the temperature is below a first threshold, the parameters are unmodified. As the temperature increases (as is normal during the course of treatment) it may eventually exceed the threshold and when this happens, the control unit can respond in several ways, by taking ameliorative measures to reduce the flow of energy into the skin, to increase the flow of energy away from the treatment site, or both. To achieve the first of these aims, the energy of each pulse may be reduced, the repetition period of the pulses may be increased, or both. To achieve the second of these aims, the control stage 24 applies power to the blower 30 to generate a stream of air to cool the skin. These measures can be applied progressively, having a greater effect as the temperature increases.

Provided that the detected temperature remains under a higher threshold, treatment can continue. However, if the temperature continues to increase, the control stage 24 will operate to suspend treatment to prevent the skin being damaged.

It must be born in mind that the aim is to control the temperature in the dermis, whereas the temperature sensor 28 provides an indication of the temperature of the epidermis. Therefore, it is necessary to calibrate embodiments of the invention. This can be achieved by experimentally identifying the detected epidermal temperatures that correspond to dermal temperatures at which ameliorative measures must be taken.

In a first enhancement, a cooling system is provided within the treatment head 12. For example, within the treatment head 12 the cooling system may operate to remove heat from the waveguide 32. Since the waveguide 32 is in contact with the skin under treatment, this has the effect of cooling the skin in contact with it, provided that the waveguide can provide a path for heat flow. To achieve this, by way of example, the waveguide is formed from quartz; a material that his good heat conductivity and a solid-state Peltier cooler 50 is applied to it as a way of removing heat from it. The Peltier cooler 50 can be activated by a signal applied to it by the control unit 10 when it detects that the temperature of the treatment site has exceeded a threshold. The waveguide 32 might also be cooled in other ways, such as by means of an air blower or by cooling liquid.

1. Dermatological treatment apparatus comprising:
a treatment head for generating intense pulses of non-laser light to an area of skin under treatment;
a controller for controlling operation of and for driving the treatment head in accordance with one or more treatment parameters;
a sensing means associated with the treatment head for detecting a condition of an area of skin under treatment;
wherein
the sensing means is operative to send a signal to the controller indicative of the detected condition, and the controller being operative to modify one or more treatment parameters in response to that signal.

2. Dermatological treatment apparatus according to claim 1 in which the controller selects initial values of the treatment parameters, and one or more of those parameters are modified during treatment in response to a signal received from the sensing means.

3. Dermatological treatment apparatus according to claim 1 in which if the controller determines that there is a risk that damage might occur at an area under treatment through an analysis of the signal from the sensing means, it is operative to modify the operation of the apparatus to minimise or remove the risk that damage will occur.

4. Dermatological treatment apparatus according to claim 1 in which the sensing means is operative to detect heat in the area under treatment.

5. Dermatological treatment apparatus according to claim 4 in which the sensing means includes a temperature sensor for measuring the temperature of the surface of the skin.

6. Dermatological treatment apparatus according to claim 5 in which the temperature sensor operated as a passive measuring device that does not introduce energy into the skin.

7. Dermatological treatment apparatus according to claim 6 in which the temperature sensor can detect infra-red radiation emitted by the skin.

8. Dermatological treatment apparatus according to claim 5 in which the temperature sensor has low sensitivity to the light of the treatment pulse.

9. Dermatological treatment apparatus according to claim 5 in which the sensing means includes an optical sensor that is operative to detect optical properties of the area of skin under treatment.

10. Dermatological treatment apparatus according to claim 9 in which the optical sensor is operative to measure the colour of the skin under treatment.

11. Dermatological treatment apparatus according to claim 10 in which the optical sensor is operative to measure the intensity of light reflected by the skin at several discrete frequencies.

12. Dermatological treatment apparatus according to claim 11 in which the optical sensor can detect one or more of blue, green, red and infra-red light.

13. Dermatological treatment apparatus according to claim 9 in which the optical sensor is operative to measure reflection of the intense light pulses that perform the treatment.

14. Dermatological treatment apparatus according to claim 1 in which the controller is operative upon detection of unfavourable conditions developing in the skin to modify treatment parameters to reduce (possibly entirely) the amount of energy that is being applied to the skin.

15. Dermatological treatment apparatus according to claim 14 in which the modified parameters include one or both of intensity of applied light pulses and repetition period of application of light pulses.

16. Dermatological treatment apparatus according to claim 1 further comprising cooling means operable to increase the rate at which energy is removed from the skin.

17. Dermatological treatment apparatus according to claim 16 in which the controller is operative upon detection of unfavourable conditions developing in the skin to operate the cooling means.

18. Dermatological treatment apparatus according to claim 1 that includes a blower for passing a stream of cold air over an area or skin being treated.

19. Dermatological treatment apparatus according to claim 1 having a contact component that is intended to be brought into contact with an area of skin under treatment, the cooling means being operative to cool the contact component.

20. Dermatological treatment apparatus according to claim 19 in which the contact component is a light guide.

21. Dermatological treatment apparatus according to claim 19 in which the cooling means includes fluid passages through and/or around the contact component.

22. Dermatological treatment apparatus according to claim 19 in which the cooling means includes a Peltier cooler.

23. Dermatological treatment apparatus according to claim 19 in which the contact component is formed of quartz.

24. Dermatological treatment apparatus according to claim 1 provided in combination with an optical compound, which compound is at a temperature below that of ambient temperature and/or below the temperature of the skin prior to commencement of treatment.

25. Dermatological treatment apparatus according to claim 1 in which the treatment head includes a flashtube for generating pulses of light.
